(19) United States
(12) Patent Application Publication
(10) Pub. No.: US 2024/0215962 A1
(43) Pub. Date: Jul. 4, 2024

(54) Device for the collection of biological material, in particular bone marrow and especially the stem cells present in the bone marrow

(71) Applicant: MDL S.r.l., Delebio (SO) (IT)

(72) Inventors: Marcello Dell'Oca, Andalo Valtellino (SO) (IT); Riccardo Dell'Oca, Morbegno (SO) (IT)

(21) Appl. No.: 18/557,570

(22) PCT Filed: May 2, 2022
(86) PCT No.: PCT/IB2022/054022

§ 371 Date: Oct. 26, 2023

(30) Foreign Application Priority Data
May 3, 2021 (IT) 102021000011150

(51) Int. Cl. A61B 10/02

(52) U.S. Cl. CPC A61B 10/025; A61B 2010/0258

(57) ABSTRACT

A device for collecting biological material, in particular bone marrow and especially stem cells, includes an external cannula for guiding an internal sampling cannula. The external cannula has a mantle wall defining an internal lumen and a distal terminal, and a plurality of openings distributed axially and circumferentially in different longitudinal and/or angular positions. The internal sampling cannula has a mantle wall that defines an internal lumen and that can be coaxially inserted inside the external cannula so that the two cannulas are mobile with respect to each other by axial and/or an angular displacement, and further has a plurality of openings that are distributed axially and circumferentially along the lateral wall in different longitudinal and/or angular positions. The holes in the external and internal cannula are distributed to enable a selective sampling in different angular and/or axial positions along the axial extension of the external cannula.

This invention refers to a device used for the collection of biological material, in particular bone marrow and specifically stem cells present in the bone marrow. This tool allows the collection of this biological material from multiple sites in a selective way, without being necessary the reposition of the device itself.

### INVENTION BACKGROUND

Bone marrow harvesting procedures are usually performed for multiple purposes, for example, diagnosing the conditions that influence different type of blood cells, to diagnose certain tumors, to obtain the marrow for transplant and/or for the collection of bone marrow stem cells. In order to collect the bone marrow, doctors generally use the pelvic bone, also known as the ileum. In fact, in most people a part of this bone is easily accessible through the lower back and is usually marked by shallow dimples on either side of the spinal column. A bone marrow aspiration procedure usually involves the passage of an aspiration cannula needle through the epithelium, the soft tissues and into the bone cortex, where the needle enters the bone. Once the needle is properly located for sampling, another cannula is placed at the sampling site which is coaxially leaded towards the first one. Therefore, an aspiration vacuum is applied to the proximal, external end of the sampling cannula. The distal terminal of the external cannula or guide cannula has several holes passing through the mantle which are located in the distal terminal section which has a set length. These holes are homogeneously distributed on the mantle wall of the distal terminal section, both longitudinally and circumferentially. Likewise, the withdrawal cannula needle, i.e., the aspiration cannula, is also present at the distal terminal, that is, the inner end of the bone and at the site of collection, a distal terminal section with several holes passing through the mantle wall, which are distributed according to a pre-established order on the mantle wall of the distal terminal, both in relation to the longitudinal position and to the circumferential position.

Furthermore, as described in document EP1641379, there is the possibility to distribute the holes on the external and internal cannula according to two interrelated orders so that, by moving the internal cannula with respect to the external one in an axial direction and/or an angular one, it is possible to match some holes of the internal cannula with holes of the external cannula having a different axial and/or angular position on the end of the external cannula in function of the axial and/or angular position of the aspiration cannula relatively to the external guide tube. Thanks to this procedure, there is no need to slide out or furtherly penetrate the external leading cannula in the bone, therefore, without causing discomfort to the patient, it is possible to collect biological material at different sampling sites distributed along the longitudinal and/or angular extension of the external cannula, simply by moving the withdrawal cannula inside the guide duct formed by the external cannula.

The type of material collected via suction through the sampling cannula usually depends on the value of the vacuum generated in correspondence of the holes in the external and internal cannula. For the withdrawal of the biological material, whose mass owns a significant component of stem cells, it is necessary to make at the holes a high suction action, i.e., to make a higher depression. This need is in contrast with the possibility of sucking the biological material through several holes that are located in different ways along the mantle area of the cannulas.

Despite the solution proposed at the state of the art allows to change the position of the withdrawal site without moving the external cannula needle with respect to the bone, this solution is still affected by some issues that make it complicated to use and they still haven't been solved in a proper way. In particular, according to an important inconvenient, the systems don't allow to verify when the internal collecting cannula has taken the second or one of the other relative positions of withdrawal with respect to the external cannula in which some holes of the withdrawal cannula are in coincidence with some holes in the internal cannula at the different positions equivalent to the different sampling sites. This problem becomes even more important when the number and distribution of the holes on the two external guide and internal cannulas have such a distribution along the distal terminal section of the two cannulas for which it is possible to open selectively leading to coincide some of the holes of the internal cannula with those of the external cannula in more than two different sampling sites each with a different position with respect to the external cannula.

Hence there is the need for an aspiration and collection device of biological material, such as bone marrow and in particular the stem cells inside of it, which could capture them in an easier, faster and more efficient way and could apply a better selection of the stem cells than other cells present in the mass of the biological material while minimizing every pain and distress associated to the patient.

### INVENTION SUMMARY

According to an executory form of this invention, a device for the collection of biological material, in particular bone marrow and especially stem cells includes:
an external guide cannula for an internal collecting cannula,
the external cannula has a mantle wall which defines an inner lumen and a distal terminal;
the mantle wall has several holes which are distributed axially and circumferentially along the side wall in different longitudinal and/or angular positions with reference to the axis of this mantle wall;
the external cannula has a proximal end, which is external to the grafting site including a grasping terminal and an opening access in the internal lumen delimited by the mantle wall,
the internal collecting cannula also has a mantle wall defining an internal lumen, and an internal cannula capable of being coaxially tucked inside the external cannula so that the two cannulas are sliding respect to each other according to an axial translation and/or an angular displacement;
the mantle wall of the internal sampling cannula has a plurality of holes which are axially and circumferentially distributed along this lateral wall in different longitudinal and/or angular positions with reference to the axis of this mantle wall;
the holes passing through the distal section of the inner cannula and those in the terminal part of the external cannula are disposed according to two interrelated orders so that the axial and/or angular displacement of the internal cannula compared to the external cannula leads some of the holes of the inner cannula in the coincident position with some of the openings of the outer cannula, which have different axial and/or angular positions along the axial and/or angular extension of the external cannula mantle wall and they communicate with the lumen of the internal cannula depending on the relative axial and/or angular position of the internal cannula with respect to the external cannula, allowing the withdrawal of the biological material through these openings and the internal cannula at different sites along the external cannula, while in each of the coincident positions the remaining openings of the outer cannula coincide with closed parts of the inner cannula mantle wall and do not communicate with the inner cannula lumen;

the internal cannula presenting a proximal end equipped with a terminal that is intended to protrude out of the lumen of the external cannula being provided for means of defined relative positioning of the internal cannula with respect to the external cannula in each of the predefined relative axial and/or angular positions in which the said openings of the inner cannula and those of the outer cannula assume one of the said positions of coincidence of at least some of the said openings of the outer cannula with at least some of the said openings of the outer cannula.

According to an executory form of this invention, the correlated distributions of holes on the external cannula and on the internal cannula are set up in such a way that there are at least three or more relative positions, at least some axial ones, in which the holes of the external cannula and of the internal cannula that come into coincidence by putting in fluid communication the internal cannula lumen with the holes of the external cannula, are coincident with as many different collecting sites along the axial and/or angular extension of the external cannula in the area of the distal terminal tract, while the relative positioning device between the distal terminal of the external cannula and the distal terminal of the internal cannula is designed in such a way to set up relative stopping positions corresponding to the said three or more positions.

An executive variant of the executory form described above provides that the means of relative positioning are made up of a series of spacer elements interposed between the distal terminal of the external cannula and the internal one. Each of these elements owns a predetermined thickness corresponding to the axial distance between two successive conditions of coincidence of the holes of the outer cannula with the holes of the inner cannula and each of which can be moved sequentially from the interposition position at a staggered position with respect to the interposition position progressively reducing correspondingly to its thickness the distance between the two distal terminals of the two external and internal cannulas.

An executory form provides that the spacer or the spacers of the series have a drawer shape radially movable from the axis of the cannulas from a position of interposition between two surfaces facing each other respectively of the proximal terminal of the external cannula and the proximal terminal of the internal cannula.

These drawer spacer elements slide on a guide jointed to the proximal terminal of the external cannula oriented in a radial direction with respect to the cannulas axe. Advantageously the impacted surfaces facing each other of the proximal terminals, or at least the stop surface of the proximal terminal of the internal cannula has a convergent inclination for a certain peripheral band along the edge of the said surface facing the spacer or while the spacer or series of spacers are made tapered as a wedge with an inclination corresponding to that of the stop surface along the front edge of the spacers facing the axis of the cannula. This executive form allows the internal cannula to be moved in steps of a certain axial length with respect to the external cannula. The length of these steps corresponds to the displacement necessary to bring into the coincident position, at each step a new group of openings on the external cannula corresponding to a new sampling site, with the corresponding openings of the internal cannula allowing the removal from the said new site.

There are different executory variants, which provide a different configuration of the spacers. For example, in an executory variant, the spacers are angularly movable between the interposition and the offset position with respect to the impacted surfaces of the distal ends of the cannulas. A further executory variant may provide that the positioning means are elastic snap type as, for example, a series of highlights along a tubular extension of the proximal terminal of the external cannula that coaxially spreads to itself and with which two teeth cooperate at the free extremities of two elastic tabs carried by a coaxial stem of the proximal terminal of the internal cannula.

Another possible variant may include positioning means consisting of a nut screw element in which the nut extends towards the screw with a coaxial threaded bushing and in which the screw is engaged. The distance between the bushing head opposite to the screw and the head of the screw is modified according to the greater or lower reciprocal screwing and also according to the pre-established relative positions between the two cannulas thanks to visible positioning notches affixed to the threaded part of the screw.

The above prove the presence of several possible alternatives for the branch technician who is able to choose between those known and present in his basic technical background and those which mainly satisfy his needs.

According to a feature that may be provided in combination with one or more of the previous claims, the distribution of holes on the external and internal cannula have different angular positions along the circumference of the cannula mantle walls in such a way that some collecting sites are positioned not only in a different axial position than the other sampling sites, but also alternatively to the axial position, only in a different angular position and/or in combination with a different axial position than the other holes even in a different angular position.

An executory form that can be provided in any combination with the previous executory forms provides that the internal cannula is closed tightly at its distal head terminal.

An executive form that can be provided in any combination with the previous also provides that the external cannula is also tightly closed at its distal head terminal.

In this case, the external cannula distal terminal is made with a perforation tip to pierce the bone wall.

In an executive variant that can be provided with any executive form of the internal cannula, it is provided that the external cannula is made open at its distal head end, with a perforation tip intended to be axially engaged inside the external cannula and which can be removed from the external cannula, the tip of which has a distal terminal like a perforation cutting edge, and at its proximal end protrudes out of the external cannula with a grasping end. The tip is equipped with some removable coupling means to the external cannula and has such a length, so that in a position coupled to the external cannula, the distal end with the cutting edge of the perforation tip escapes from the distal end of the external cannula.

According to an executory form, a further feature that can be provided in any combination or sub combination with one or more of the previous executive forms, is that the dimension of the holes located along the cannula mantel of the internal and external cannula is variable with reference to the position of the holes along the mantle surfaces and in particular they follow the axial position of these openings with respect to the distal ends of the external cannula and/or of the internal cannula in such a way that they can compensate for the variations in the depression aspiration alteration created in correspondence with the different holes in accordance with their axial position along the cannulas that would occur if all the holes had the same dimension.

This feature makes it possible to ensure that for each sampling setting at each of the sampling sites, the depression generated is high and enough to aspirate stem cells more predominantly than the other cells that together are part of the biological material.

Indeed, it is well known that, in order to maximize the collection of stem cells compared to the whole aspirated mass it is necessary to make a major depression than a preestablished value which is set in advance before the product purchase.

According to another profitable feature, the proximal terminals of the external and internal cannula are equipped with cooperative end-of-stroke collisions that delimit the displacement of the internal cannula with respect to the external cannula between two extreme positions of maximum introduction of the inner cannula into the outer cannula and maximum extraction of the inner cannula with respect to the outer cannula. This procedure avoids that the internal cannula could be pushed too much inside the insertion site with respect to the distal terminal of the external cannula causing pain or damage. Furthermore, it prevents the accidental sliding of the internal cannula from the external, for example when you want to separate the connection between the distal terminal and the suction devices.

The suction devices can be of any type, such as manual or motorized means. In the first case it is possible to use a syringe or a plunger pump, while in the second case it is possible to use motorized pumps typically used in the biomedical field, like peristaltic pumps or aspirations by means of venturi-generated vacuum or other types of pumps. As said above, the particular building of the device according to this invention allows selective and relative movement of the internal cannula over the external one and allows the device to be configured in different collecting ways of the biological material, where at least one of the holes in the lateral wall of the inner cannula can be aligned with one of several holes in the lateral wall of the outer cannula so that the biological material, i.e., the bone marrow cells, or predominantly stem cells present in the bone marrow can be aspirated into the inner lumen of the inner cannula from different radial and/or longitudinal positions outside the outer cannula without changing the position of the outer cannula. Therefore, the selective movement between the inner cannula and the outer cannula can change the position of the extraction holes by aligning different holes in the inner and outer cannulas.

An advantage of this invention consists in having defined relative positions between the two cannulas corresponding to the different sampling sites, without the constant monitoring of the user, making the process safer and faster. This invention allows a doctor to collect biological material from various locations in a wider area without making necessary the reposition of the collecting device. Once made the withdrawal from a collecting site adjacent to a first suction hole, the doctor may manipulate the device in order to close this aspiration hole and open a second suction hole coinciding with a different area, all changing the location of the collecting site.

The choice of the different dimension of the holes according to their axial and/or even radial position allows to prevent a decrease of the depression along the longitudinal extension of the cannula and it guarantees that the depression always has high values in any collecting site of the different axial positions along the cannulas making it possible a prevailing collection of stem cells among the biological material.

### BRIEF DESCRIPTION OF THE IMAGES

Images 1 and 2 are respectively a lateral elevation view and a perspective view of the external cannula.

Images 3 and 4 are respectively a lateral elevation view and a perspective view of the internal cannula.

Images 5 and 6 are respectively a lateral elevation view and a perspective view of the perforation tip.

Images 7 and 8 are respectively a lateral elevation view and a perspective view of the external cannula with the working perforation tip.

Image 9 is a perspective view of the external cannula with the working external cannula.

Images 10 to 12 show respectively a view in lateral elevation of the internal cannula inserted into the external one, an enlarged detail respectively in a lateral elevated view and in a section according to a diametral plane of the terminal, distal section with the opening coupled according to the first operational setting.

Images 13 to 15 show respectively a view in lateral elevation of the internal cannula inserted into the external one in the second of the two operational settings, an enlarged detail respectively of the diametral plane of the distal, terminal section with the opening in the same position of the second operational setting.

Images 16 and 17 show an enlarged detail of the terminals on the proximal ends of the two working cannulas and respectively with the relative positioning means of the two cannulas made up of a detachable spacer of the two distal terminals in the two working positions.

Images 18 and 19 show variants of the relative positioning middles of the two cannulas which allow the sequence movement in predefined positions in a continuous way.

### DETAILED DESCRIPTION OF THE INVENTION

In this description and in the claims the terms openings and holes are considered equivalent and synonymous.

In addition, in this description and in the claims, the term “depression” technically indicates a difference in pressure between the current pressure on the collecting site and the pressure generated inside the collecting cannula and in correspondence with the collecting holes. Obviously, when the description refers to the term aspiration it means that this effect is caused by the creation of a depression, i.e., a difference in negative pressure between the collecting site environment and the inner part of the collecting cannula and vice versa the indication of said difference in pressure it is meant to communicate a generation of an aspiration of the mass of the biological material in the withdrawal site, hence in the adjacent area to the withdrawal openings which are in communication with the internal cannula.

In the images is illustrated an executory form and some of its versions. These are considered executory examples which aim is to concretely explain the inventive teaching of this invention and to indicate an implementation way to the technician. Further implementation variants are the result of the invention application to different implementation solutions that are in the baggage of the branch expert.

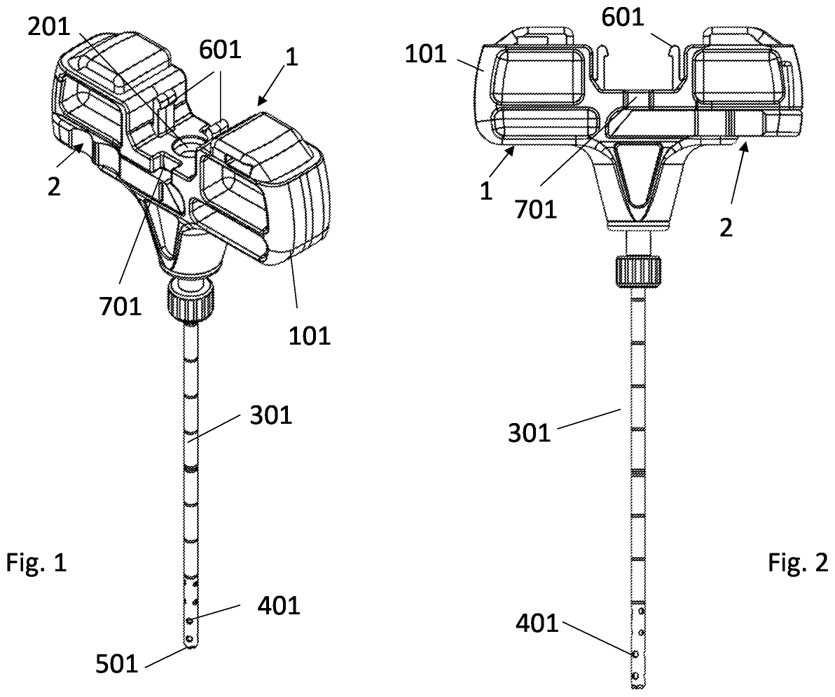

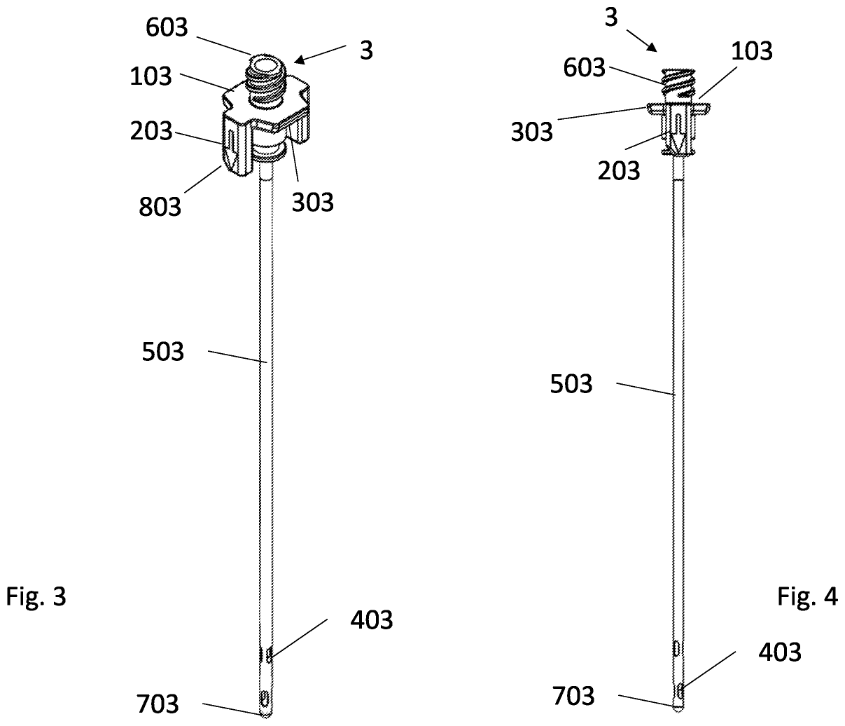

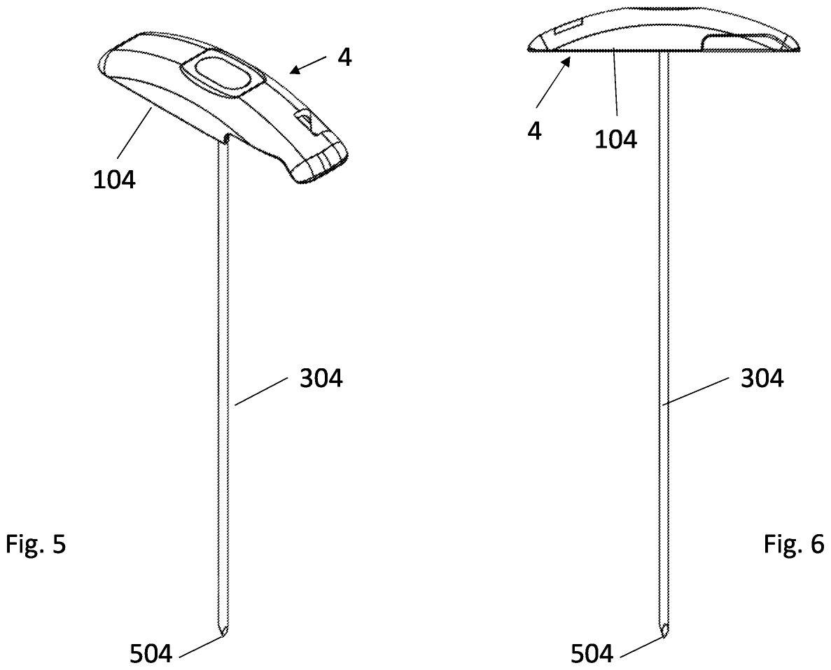

Images 1 to 17 illustrate an executory form of the invention. The device is composed by three parts: one of these is an external cannula needle 1 which is introduced into the bone carrying a distal terminal 501 of the cannula 301 in the collecting site, for example in the marrow bone inside the bone. This operation is made by combining the external cannula 1 with a perforation and insertion tip 4 (FIGS. 5 and 6) which includes a stem 304 ending with a perforation tip 504 on distal terminal and on the opposite end it has a grasping head 104, which is allocated to stay outside of the corresponding extremity 101 of the external cannula 1.

This grasping head 104 is configured to be combined with a terminal grasping head 101 provided at the proximal terminal of the external cannula needle 1 which is used as a guiding device during the insertion step into the bone tissues and inside the bone itself.

In the executory form illustrated, the two proximal terminal 101 and 104 have reciprocal battling surfaces, therefore, by applying forward pressure in the bone at the end 104 of the insertion tip, this pressure is also transmitted to the end 101 of the external cannula 1.

According to a feature, the length of the perforating tip is defined to such an extent that during the mutual battling condition of the grasping terminals 101, 104, the extremity 504 i.e., the perforation sharp tip, protrudes outside the open terminal 501 of the guide cannula 301 of the external cannula needle 1.

According to an executory form, the leading edge of the guiding cannula 301 in correspondence with the distal terminal 501 can be shaped in order to make a cutting action so that it can assist the action of the perforation tip and/or, if necessary, provide the discharge of the cutting material outside the guiding cannula section 301.

The external cannula needle 1 once positioned is separated from the insertion tip that is simply removed from it, thus freeing the internal light for the insertion of an internal collecing cannula as seen in images 3 and 4.

The collecting cannula 3 has a tubular body 503 which is closed at its distal head terminal for insertion into the cannula 301 of the external cannula 1. At the proximal terminal of the cannula there is a terminal 101 of the external cannula needle 1 and in the operating condition it protrudes outside the guiding cannula 301 on the side of the proximal terminal of it.

According to the illustrated executory form, the internal sampling cannula is sliding inside the external cannula in axial direction. As will be seen below, the example above does not imply the possibility of angular displacement, however this limitation it is not considered a limit and in an executory form it is possible that the internal cannula 3 could rotate around its axis and relative to the driving cannula 301 in combination with the possibility of a translation.

As is evident from the images, the external cannula needle 1 has at its distal end a terminal section of a predetermined length along which are distributed axially and angularly some holes indicated with number 401. These openings are passageways and have a pre-established passage light.

Similarly, the collecting cannula 503 has at its distal end 403 some holes too that are distributed on the wall of the internal cannula 503 both longitudinally (axially) and angularly.

Between the cannula 1 and the internal cannula 3 there are further relative guide organs and some end of stroke stops in two opposite axial positions of the internal cannula 3 with respect to the external cannula 1.

These stops have cooperating radial walls in the direction of introduction and a pair of retaining teeth in the direction of extraction on the terminal 101 of the external cannula that engages radial fins 203 of the terminal of the cannula internal 3.

These retaining teeth are provided at their extremity of vertical tabs 601 which are elastically detachable to allow the introduction of the terminal 103 of the internal cannula 3 in the position interposed between the stop surface of the end of stroke insertion of the internal cannula 3 in the external guide cannula 1 and the retaining teeth on the fins 601.

According to another feature that can be provided or not, the two terminals 101 and 301 have cooperating means of axial sliding guide anti-relative rotation.

According to this executory form, the terminal 103 of the internal cannula 3 has at least one pad or at least one axial slide 203 which is intended to slide with an axial guide 701 provided on the terminal 101 of the external cannula 1.

In the executory form illustrated this guide is made up of a sliding groove obtained in the body of the terminal 101 of the external cannula 1 and in which works the sled cantilevered in position coinciding with the groove from the terminal 103 of the internal cannula 3. The groove is opened at its upper and lower head ends.

Thanks to this configuration, when the internal cannula 3 is inserted into the external cannula 1, the pad 203 of the internal cannula 3 automatically matches with the corresponding guide 701, the skid and the guide laid down in the position between them.

According to an executory form, the terminals 101 and 103 of the external cannula 1 and the internal cannula 3 are equipped respectively with at least two guides arranged angularly staggered with reference to the axis of the cannulas and at least two slides positioned angularly corresponding with the two guides. In particular, the two guides and the slides 701, 203 are positioned two diametrically opposite each other.

When the terminal 101 of the external cannula needle 1 has a T-shape with a transverse grasping branch extending along a diametrical plane, then at least two sleds and at least two guides 203, 701 diametrically opposed to each other are aligned along a diametrical plane perpendicular to it along which extends the terminal of grab 101 with a T shape. The terminal 103 of the internal cannula 3 is extended with a sealing connection terminal 603, with a suction device (vacuum generator) but not limited to the connector of the type Luer Lock shown in the images.

According to an additional feature the mantle wall 503 has some holes 403 that are axially and angular distributed according to a predetermined design.

The holes 401 provided on the distal terminal of the external cannula needle 1 are also distributed according to a predetermined axial and angular design of the cannula. The two distribution designs are related to each other in such a way that, for different relative axial positions between the internal sampling cannula 3 and the external guide cannula 1, some of the 401 openings of the external cannula 1 are in the coincident position with at least some of the holes 403 of the internal cannula in order to communicate with the duct defined by that internal cannula 3 and intended for the passage of the biological material sampled. Whereas others remain coinciding with parts of the internal cannula mantle wall, i.e., closed. Changing the relative axial position between the two cannulas 1 and 3, the position of the holes 401 that coincide with the holes 403 and that communicate with the internal cannula 3 and the location of the biological material collection site along the external cannula changes too.

It should be remembered that this description is limited to an executory example in which the two internal and external cannulas 3, 1 are only translatable in their axial direction, or rather, the internal cannula is moved axially inside the internal cannula, while the two cannulas can't rotate all around their axis.

Such solutions seem more appropriate, however, there are different variants that provide the combination of the axial translation displacement with an angular shift of the internal cannula 3 with respect to the external cannula 1. This combination of translational and rotary movement complicates the distributions of the holes 401 and 403, for which this example is limited to this solution that allows the only axial translation of the internal cannula 3 with respect to the external cannula 1 and also to an executory form in which the cannula 3 can only adopt two different axial positions with respect to the external cannula 1. Then it is possible to provide more than two relative positions between the two cannulas 1 and 3 and therefore more than two different sampling sites along the axial extension of the external cannula 1.

In images 10 to 15 is shown the combination of the external cannula needle 1 of the internal cannula 3 guide, respectively in the two sampling positions from two sampling sites provided in different positions along the axis of the cannula needle. For these the sampling is activatable thanks to the translation of the internal cannula 3 from a position of maximum introduction and a second position of partial extraction for an axial extraction stroke with a predefined length and in combination with the distributions between their correlated holes 401 and 403 in the outer cannula mantle wall 301 and in the mantle wall 503 of the inner cannula.

Referring to images 11 and 12, the distal end of the mantle wall 301 of the external cannula 1 has four crowns of apertures distributed angularly along the circumference of the mantle wall and they have a preestablished axial distance between them.

Each crown of openings may have at least two or more openings distributed along the circumference and the angular position of the openings may be identical or different from crown to crown and this angular position may be different and/or identical for groups of one or more adjacent crowns of openings.

In this specific case two crowns of adjacent openings have an identical angular position of the openings, while each pair of crowns has different angular positions that are staggered between them, the first and the second crown of openings, starting from the distal terminal 501 have an identical angular position of the openings, while the third and fourth crown of openings have identical angular position, but different from the first and second crown of openings. The openings visible in FIGS. 11 and 12 of the first crown are indicated with 411 and 421, those of the second with 451 and 461, those of the third with 431 and 441 and those of the fourth with 471 and 481.

The axial distance between the first and third crown of openings is substantially identical to the axial distance between the second and fourth crown of openings and the two pairs i.e., the first and the third and the second with the fourth crown of openings, which are clearly axially spaced to the same extent, while the openings of the first crown and those of the second crown are angular in relation to those of the third and fourth crown of openings.

Clearly, the openings 403 present in the terminal section of the inner cannula 3 mantle wall are distributed along two crowns of openings indicated in the images 11 and 12 respectively with 413 and 423 and with 433 and 443. The two crowns of openings respectively with the openings 413, 423 and 433, 443 are axially spaced correspondingly to the axial distance of the first from the third crown of openings 411, 421 and 431, 441 in the mantle surface of the external cannula and also corresponding to the axial distance between the second and fourth crown of openings 451, 461 and 471, 481.

According to the present invention this measure of the distance between the openings in the axial sense is taken relative to the central point of the diameter of the openings parallel to the axis of the cannula 1, 3.

Each crown of openings 413, 423 and 433, 443 has an angular distribution of these openings which corresponds to the angular arrangement of the openings respectively of the first and second crown of openings and of the third and fourth crown of openings in those of the external cannula 1 mantle wall 301. In fact the crown of openings 413, 423 closest to the distal terminal of the inner cannula 3 has an angular distribution of the openings corresponding to the holes in the mantle wall 301 of the external cannula 1 provided for the first and second crown of openings, while the second crown of openings, which is the farthest from the closed distal terminal of the inner cannula 3 has an angular distribution of the openings corresponding to the openings in the mantle wall 301 of the external cannula 1 provided for the third and fourth crown of openings.

In the condition of maximum introduction of the internal cannula 3 in the external cannula 1 in the images 10 to 12, the openings 413, 423 and 433, 443 of the two crowns of openings of the internal cannula are coincident with the openings 411, 421 and 431, 441 of the first and third crown of openings in the mantle wall of the external cannula 1. Therefore, the biological material is collected all around the external cannula 1 in the two areas axially coinciding with the first and the third crown of openings 411, 421 and 431, 441.

In images 13 to 15, is shown the combination of external cannula needle 1 with internal cannula 3 inserted in the second position of maximum extraction of the inner cannula 3 with respect to the external cannula 1. The inner cannula is retracted axially for a distance corresponding to the distance between the first and second crown of openings 411, 421 and 451, 461 in the external cannula mantle wall 301 and corresponding to the distance between the third and the fourth crown of openings 421, 431 and 471, 481.

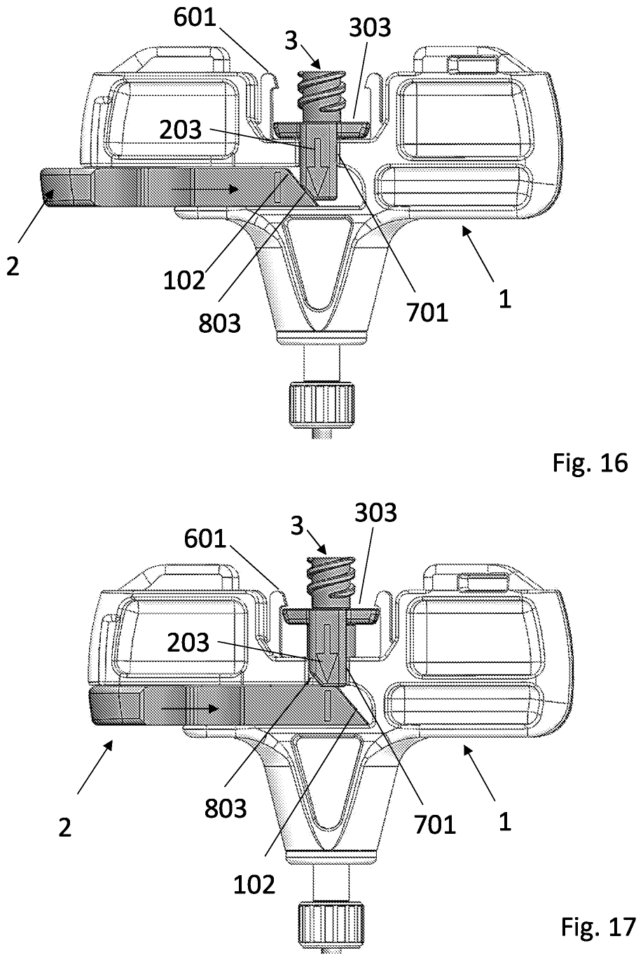

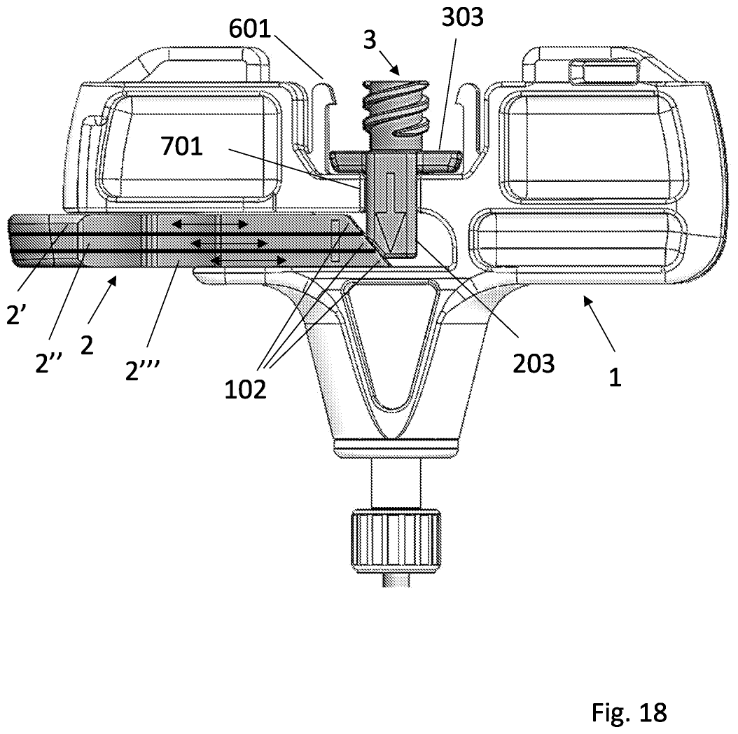

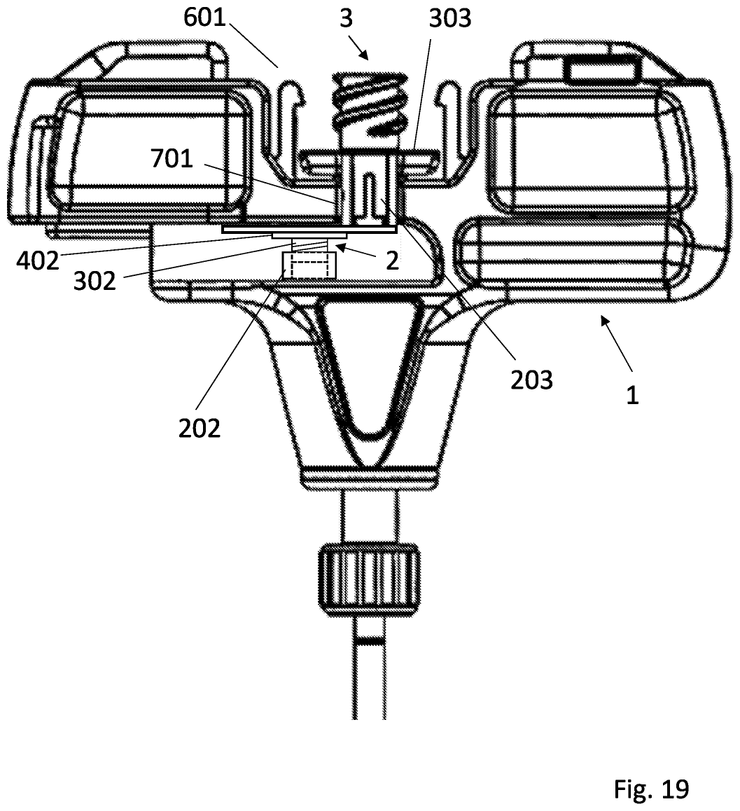

As is evident from FIGS. 14 and 15, this axial displacement in the direction of extraction of the inner cannula 3 implies that the openings 413, 423 and 433, 443 of the two crowns of openings in the mantle wall of the inner cannula 3 coincide with the corresponding openings 451, 461 and 471, 481 of the second to fourth crown of openings in the mantle wall 301 of the outer cannula 1.

It is worth noting how the openings 411, 421 of the first crown of openings of the outer cannula 1 are located below the closed distal end 703 of the inner cannula 3 and therefore do not make any suction action on the material through those openings.

The openings of the third crown are coinciding with a part of the wall of the inner cannula closed mantle 503 so that they are not working for the collection because they don't communicate with the inside of the inner cannula 3.

It is evident that, following the same logic, it is possible to foresee a greater number of pairs of crowns of openings along at least the mantle wall 301 of the external cannula, maintaining only the two crowns of openings of the inner cannula, or you can also change the distribution so that the internal cannula 3 also has more than 2 crowns of openings. The distances can then be modified to have several different sampling sites distributed all along the axial extension of the external cannula 1.

According to the present invention, the device also provides, in combination with the two distributions of openings in the external cannula 1 and in the internal cannula 3, a positioning device of the internal cannula 3 compared to the external cannula 1 that allows to move the internal cannula 3 with respect to the external one 1 for the translation stroke of length corresponding to the variation of the condition of coincidence of some openings of the external cannula 1 with some openings of the internal cannula 3 and which are provided in different axial positions depending on the relative axial position of the two tubes.

In the illustrated and described form which has only two relative positions of the internal cannula 3 with respect to the external cannula 1, which are used for the extraction of biological material from two different sampling sites having different positions with respect to the axial extension of the external cannula, between the proximal terminals 101 and 103 of the external cannula 1 and of the internal cannula 3, are provided relative axial positioning devices of the two cannulas in the two pre-established axial positions and providing a stable end-of-stroke position respectively in those two positions, in such a way that the user does not have to find by approximation and/or by tests and errors the correct relative position of the two tubes 1, 3, which guarantees the coincident position of the openings for sampling from one of several sampling sites distributed along the axial extension of the external cannula. With reference to this executory form, the axial relative positioning device of the cannula 3 with respect to the cannula 1 in the two positions referred to images 10 to 15 consists of a spacer of thickness corresponding to the stroke between the said two positions, or at the distance between the first crown of openings and the second crown of openings 411, 421 and 451, 461 at the distal terminal of the outer cannula 1.

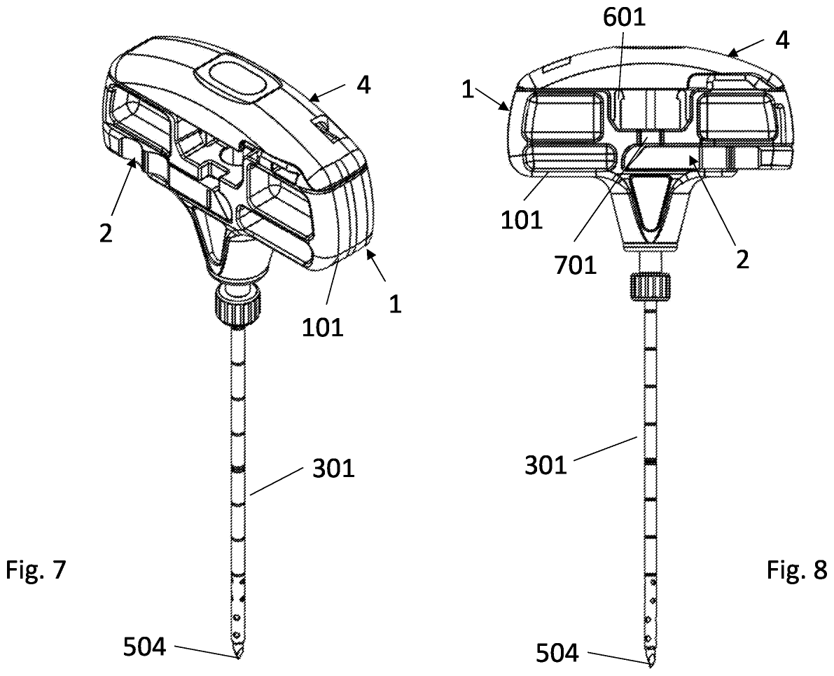

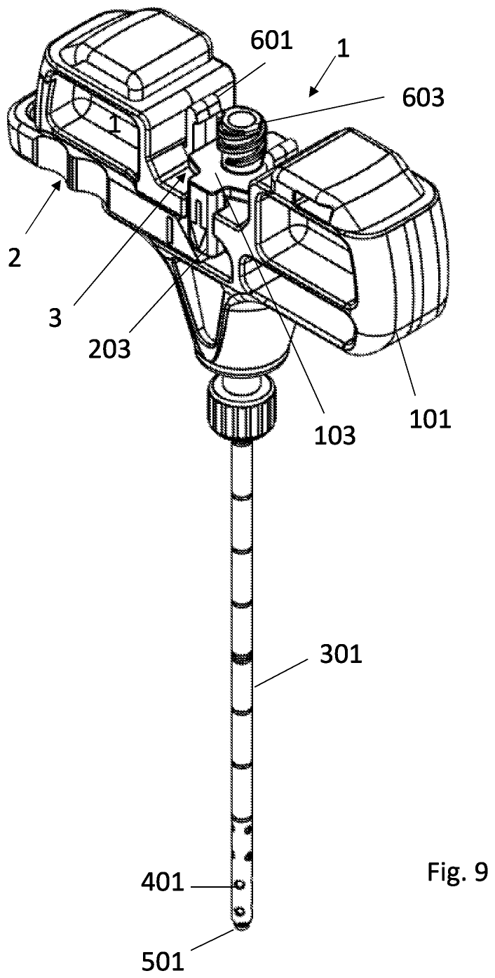

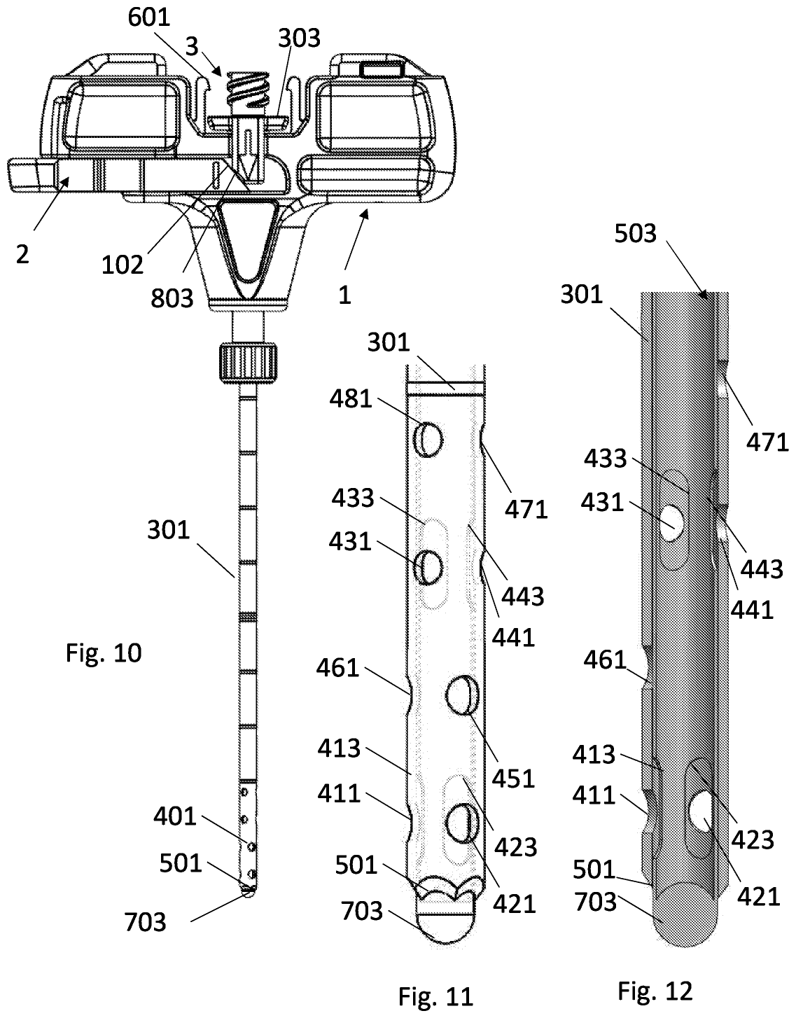

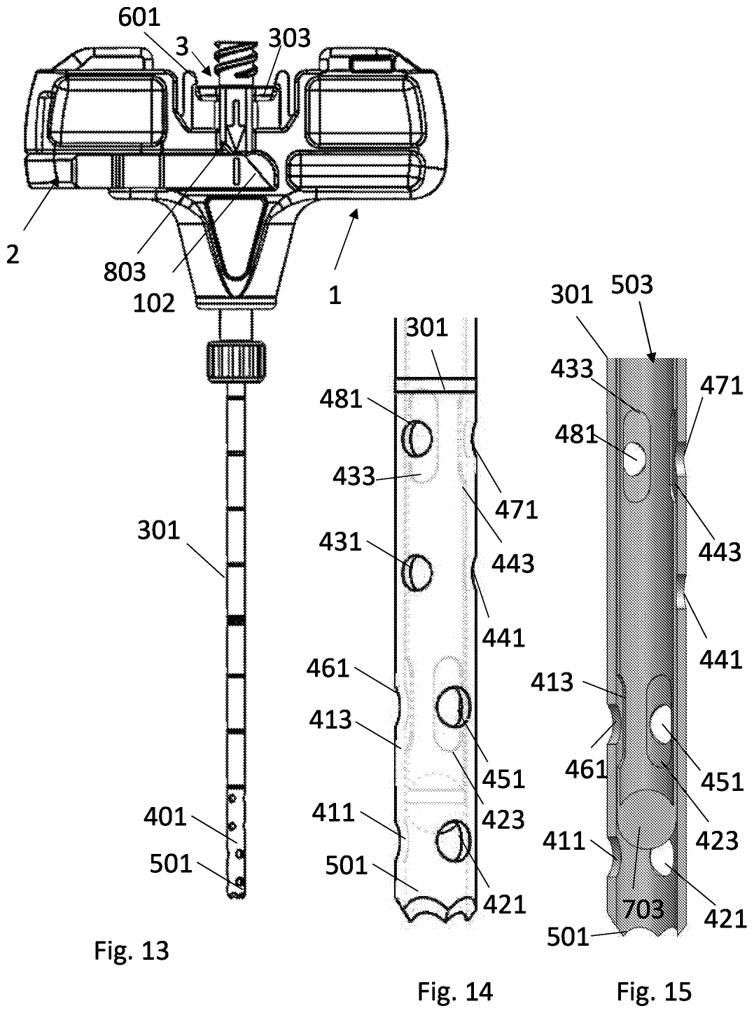

This spacer 2 is an end-of-stroke stop surface cooperating with the terminal 103 of the internal cannula 3 which can be carried by translation in the radial direction of the cannulas 1, 3 in a position of interference with the said terminal 3 of the internal cannula 3 where the terminal 103 is struck against a surface of the spacer holding the internal cannula 3 in a retracted position behind the thickness of the spacer and in a non-interference position where terminal 3 can arrive at the end of the insertion of the internal cannula 3 into the external cannula 1 where it is stopped by the end-of-stroke surfaces 303 of terminal 3 that collides with facing surfaces of the terminal 101. Image 16 shows an intermediate position of the spacer 2 in which, thanks to an inclined plane 102 facing a corresponding inclined plane 803 of terminal 3 of the internal cannula and in particular of the peripheral edge of the slides 203, the passage from one axial position to another of the two axial positions provided for the internal cannula 3 takes place in a progressive and controlled way. Pushing the spacer 2 according to the direction of the arrow, the inner cannula 3 is progressively raised from the maximum insertion position of cannula 1 as in FIGS. 10 to 12 and is then brought to the maximum extraction position corresponding to the position of FIGS. 13 to 15 to which it corresponds the end-of-stroke position of the insertion spacer 2 below the skid 203.

Image 18 shows an executory variant that involves four displacement steps between the internal cannula 3 and the external cannula 1 including a maximum insertion position in the cannula 3 in the cannula 1 and a maximum extraction position of the cannula 3 from the cannula 1 and two intermediate positions distributed for axial displacement steps of identical distance. In this case, the spacer 2 consists of three spacers 2′, 2″, 2′″ which slide radially independently of each other and which have a tapered terminal with one side of the head inclined so as to form together the inclined plane 803. Obviously, in this case, the number of crowns of openings in the mantle wall 301 of the external cannula 1 and their distance must be changed correspondingly to the axial displacement steps of the preestablished length of the cannula 3 with respect to the cannula 1.

According to an executory variant, the displacement of the spacer may not be a radial translation, but a rotation around an axis parallel to the axis of the cannulas 1, 3, which has an axial thickness increasing in the same angular direction. A further executory variant is illustrated in image 19, here the system of controlled variation of the relative axial position between the internal cannula 3 and the external cannula 1 is progressive and continuous and, it includes a spacer of screw and nut type, which are interposed between two facing surfaces of the terminal 101 of the external cannula 1 and the terminal 103 of the internal cannula 3. A threaded screw 302 has a head radially widened 402 with which it strikes against the lower head side of the pads 203 or another surface of the terminal 3. The screw 302 is made up with a basic bushing which rests against a stop surface of the terminal 101 of the external cannula 1. By rotating relatively between them the screw 302 and the threaded bushing 202 are constantly varied and progressive in the distance between the screw head 402 and the threaded bushing base 202 so that the end of the inner cannula 3 is moved progressively and axially with respect to the terminal 101 of the external cannula 1 in the direction of removal or reciprocal approach depending on the direction of rotation and the internal cannula 3 is consequently axially translated for a corresponding stroke in and out compared to the external cannula 1.

The correct relative positions corresponding to the various coincidence positions can be indicated thanks to notches and/or colours like the screw 302.

It should be noted that the executory forms shown are all manually operated, but that the same executory forms can also be combined with motorized actuators.

According to another feature that does not appear evident from the images, the openings provided for in the mantle wall 301 of the external cannula 1 and/or even the openings provided for in the mantle wall 503 of the internal cannula 3 have a passage light of different sizes depending on their axial position according to the end of the external cannula 1, in order to compensate for pressure difference losses occurring along the cannulas.

Furthermore, the openings' passage lights are dimensioned in such a way that the depression and so the aspiration exerted by each opening is such as to allow to maximize the aspiration from the mass of biological material, i.e., the bone marrow of some cell types, or stem cells, so as to obtain a bigger content of those cells in the total mass of the material collected.

It is worth considering here that as shown in images 10 to 15, the openings 403 provided in the mantle wall of the inner cannula 3 are made elongated in the axial direction. First, this prevents positioning tolerances from generating conditions of partial coincidence of the openings of the external cannula 1 with those of the internal cannula 3. Then, when considering a progressive control of the relative axial positions between internal cannula 3 and external cannula 1, establishing, for example, a width variation of the internal cannula openings 3, that is, their light in angular direction, which size varies from a maximum size at one end of the opening to a minimum size at the opposite end and where the maximum size is substantially the size of the openings in the mantle wall 301 of the external cannula 1, while the minimum size is at least a fraction of the corresponding size of the openings in the mantle wall 301, it is possible to change the passing light between the outside of the external cannula 1 and the inside of the internal cannula 3, thus modifying the progressive variation of the openings passage lights, with reference to their position along the axial extension of the cannulas 1, 3, is the depression generated at the openings of the external cannula 1. This allows the device to be calibrated to make it suitable for a selectively optimized collection of different cells present in the mass of biological material such as in particular in the bone marrow and/or inside the bone.

The invention claimed is:

1. A device for collection of biological material, comprising:
an external guide cannula for an internal sampling cannula,
wherein:
the external cannula has a first mantle wall which defines an inner lumen and a distal terminal;
the first mantle wall has a first plurality of openings which are distributed axially and circumferentially along a first side wall in different longitudinal and/or angular positions with reference to an axis of the first mantle wall;
the external guide cannula has a proximal end, external to a grafting site, the proximal end comprising a grasping terminal and an inlet opening in the inner lumen bounded by the first mantle wall;
the internal sampling cannula has a second mantle wall which defines an internal lumen, the internal sampling cannula being configured to be coaxially inserted inside the external guide cannula so that the external guide cannula and the internal sampling cannula are mobile with respect to each other by axial translation and/or an angular displacement;
the second mantle wall of the internal sampling cannula has a second plurality of openings which are distributed axially and circumferentially along the second mantle wall in different longitudinal and/or angular positions with reference to an axis of the second mantle wall;
the openings in the second plurality of openings passing through a distal part of the internal sampling cannula and the openings in the first plurality of openings defined in a distal part of the external guide cannula are distributed according to two interrelated orders so that the axial translation and/or angular displacement of the internal sampling cannula with respect to the external guide cannula leads some of the openings of the inner sampling cannula to be in a coincident position with some of the openings of the external guide cannula, which have different axial and/or angular positions along an axial and/or angular extension of the first mantle wall and are communicating with the lumen of the internal sampling cannula depending on a relative axial and/or angular position of the internal sampling cannula with respect to the external guide cannula;
the biological material can be extracted through some of the openings in the internal sampling cannula from different sites along the external guide cannula, the remaining openings in the external guide cannula coinciding with closed portions of the second mantle wall and failing to communicate with the inner lumen of the internal sampling cannula; and
the internal sampling cannula has a proximal end that is equipped with a terminal configured to protrude outwards from the lumen of the external guide cannula and enable a relative positioning of the internal sampling cannula relative to the external guide cannula in predefined relative axial and/or angular positions in which the openings of the internal sampling cannula and the openings of the external guide cannula coincide with some of the openings of the outer external guide cannula with at least some of the openings of the internal sampling cannula.

2. The device, according to claim 1, wherein distributions of the openings on the external guide cannula and on the internal sampling cannula are arranged to provide three or more relative positions in which holes of the external guide cannula and of the internal sampling cannula, which align to put in communication the lumen of the internal sampling cannula with the holes of the external guide cannula, coincide with as many different sampling sites along an axial and/or angular extension of the external cannula in a distal terminal portion of the external guide cannula, and wherein relative positioning means between the distal terminal portion of the external guide cannula and a distal terminal portion of the internal guide cannula is designed to provide relative stopping positions corresponding to the three or more positions with translation steps between the external guide cannula and the internal sampling cannula of predetermined amounts. or in a progressive and continuous manner.

3. The device, according to claim 2, wherein the relative positioning means consist of a series of spacer elements interposed between the distal terminal of the external guide cannula and the distal terminal of the internal sampling cannula, wherein each of the spacer elements has a predetermined thickness corresponding to an axial distance between two successive conditions of coincidence of the holes of the external guide cannula with the holes of the internal sampling cannula, and wherein each of the spacer elements is movable in a progressive sequence from an interposition position to a staggered position with respect to the interposition position, progressively reducing a distance between the distal terminals of the external guide cannula and internal sampling cannula.

4. The device, according to claim 1, wherein distributions of the openings on the external guide cannula and the internal sampling cannula have different angular positions along a circumference of the first and the second cannula mantle wall in such a way that some sampling sites are placed not only in a different axial position compared to other sampling sites, in a different angular position, and/or in in the different axial position than other openings even in the different angular position.

5. The device, according to claim 1, wherein the internal sampling cannula is sealed at a distal head end thereof.

6. The device, according to claim 1, wherein the external guide cannula is open at a distal head end thereof, wherein a perforation tip can be axially engaged inside the external guide cannula and removed therefrom, wherein the perforation tip has a distal end configured as a perforation cutting edge and a proximal end configured to protrude out of the external guide cannula with a grasping end, and wherein the perforation tip is equipped with removable coupling means to the external cannula and has such a length, so that in a position coupled to the external cannula, the distal end with the perforation cutting edge of the perforation tip escapes from the distal end of the external guide cannula.

7. The device, according to claim 1, wherein passage lights of the openings along the first and/or the second mantle walls are variable with reference to an axial position of the openings with respect to the distal ends of the external guide cannula and/or of the internal sampling cannula, so as to compensate for variations in aspiration depression generated at different openings as a function of an axial position of the openings along the external guide cannula and the internal sampling cannula which would arise if the openings all had passing lights of identical dimensions.

8. The device, according to claim 1, wherein a proximal grasping terminal of the external guide cannula and a proximal terminal of the internal sampling cannula are equipped with cooperative end-of-stroke stops that delimit a displacement of the internal sampling cannula with respect to the external guide cannula between two extreme positions of maximum introduction of the internal sampling cannula into the external guide cannula and maximum extraction of the internal sampling cannula with respect to the external guide cannula.
